(19) United States
(12) Patent Application Publication
(10) Pub. No.: US 2022/0144752 A1
(43) Pub. Date: May 12, 2022

(54) WATER-SOLUBLE NAPHTHOQUINONE DERIVATIVE COMPOSITION AND METHOD FOR PRODUCING SAME, WATER-SOLUBLE COMPOSITION FOR CONTROLLING HARMFUL ALGAE, METHOD FOR CONTROLLING LARGE-SCALE HARMFUL ALGAE, AND AUTOMATION SYSTEM FOR AI-MONITORING, REMOVING, AND PREVENTING LARGE-SCALE HARMFUL ALGAE

(71) Applicant: WATER-BIOTECH, Seoul (KR)

(72) Inventors: Hae-dong SEO, Seoul (KR); Myung-soo HAN, Seoul (KR)

(21) Appl. No.: 17/432,430

(22) PCT Filed: Jan. 22, 2020
(86) PCT No.: PCT/KR2020/001090

§ 371 Date: Aug. 19, 2021

(30) Foreign Application Priority Data
Feb. 19, 2019 (KR) 10-2019-0018998

(51) Int. Cl. C07C 225/30; A01N 33/10; A01P 13/02; A01N 25/12; B64C 39/02

(52) U.S. Cl. CPC C07C 225/30; A01N 33/10; A01P 13/02; B64C 2201/146; B64C 39/02; B64C 2201/12; A01N 25/12

(57) ABSTRACT

Provided are a water-soluble naphthoquinone derivative composition and a method for preparing the same, a water-soluble composition for controlling harmful algae, a method of controlling large-scale harmful algae, and an automated system for artificial intelligence monitoring, removal, and prevention of large-scale harmful algae. According to one embodiment, a method of preparing a water-soluble naphthoquinone derivative composition includes reacting a 1,4-naphthoquinone compound with N,N-diethylethylenediamine to obtain an intermediate product of [Chemical Formula 2]; and reacting an intermediate product of [Chemical Formula 2] with hydrochloric acid to obtain a compound of [Chemical Formula 1]. In addition, the water-soluble naphthoquinone derivative composition represented by the following [Chemical Formula 1] is proposed. In addition, a water-soluble composition is proposed for controlling harmful algae, a method for controlling large-scale harmful algae using the water-soluble composition for controlling harmful algae, and an automated system for artificial intelligence monitoring, removal, and prevention of large-scale harmful algae.

### BACKGROUND OF THE DISCLOSURE

### Field of the Disclosure

The present disclosure relates to a water-soluble naphthoquinone derivative composition and a method for preparing the same, a water-soluble composition for controlling harmful algae, a method of controlling large-scale harmful algae, and an automated system for artificial intelligence monitoring, removal, and prevention of large-scale harmful algae. More specifically, the present disclosure relates to a water-soluble naphthoquinone derivative composition with improved water solubility that can be directly used in on-site water without an organic solvent to remove harmful algae and a method of preparing the same, a water-soluble composition for controlling harmful algae, a method of controlling large-scale harmful algae, and an automated system for artificial intelligence monitoring, removal, and prevention of large-scale harmful algae.

### Related Art

In temperate regions such as Korea, since the abundance of nutrients in eutrophic aquatic ecosystems creates an environment favorable for the growth of primary producers such as phytoplankton, the explosive generation of harmful algae is repeated every year (Ministry of Environment 2012). As changes in hydrological environment and climate warming, such as the increase in residence time of water due to the construction of multiple dams or weirs even in domestic rivers or streams progress, the occurrence of mass proliferation of harmful algae was further accelerated. In particular, green algae became a social issue in the whole of Namhan River and Bukhan River, including the Nakdong River to the extent that new word “green algae latte” was coined.

On the other hand, damage caused by harmful algae is not limited to freshwater ecosystems, and red tide damage is serious in the ocean. Harmful algae organisms have the same toxicity as PSP, DSP, ASP, etc. The toxicity concentrated in fish and shellfish have resulted in the death of not only humans who eat fish and shellfish, but also birds and mammals, pets and livestock on the waterfront or beach.

Research is being conducted in various fields to control the phenomenon of green algae and red algae not only in Korea but also around the world, but It is not possible to control large-scale harmful algae with the technology commercialized so far.

Conventionally, in order to control harmful algae, a method of directly spraying chemical algicides such as copper, Reglone A, potassium permanganate, chlorine, and Simazine, coagulants such as clay, and phosphorus reducing treatment agents such as Phoslock® onto a water system has been used. Since hardly decomposable substances such as heavy metals belonging to these chemical methods have secondary contamination problems such as occurrence of toxic phenomena caused by bioaccumulation through the food chain, and coagulants require secondary treatment to recover the sludge, there is a cost for manpower and equipment operation, and in particular, in the case of sedimentation agents such as loess, there is a limit in which the disturbance of the benthic ecosystem is expected. Due to the disadvantages of each of these technologies, these technologies are not only difficult to apply in the field, but can also be threatened to the stability of the ecosystem.

Meanwhile, in order to overcome the above problems, a technology for a composition for controlling harmful algae has been proposed by making naphthoquinone derivatives with algal effect on harmful algae and using the naphthoquinone derivatives. As suggested in the patents to be described later, these conventional naphthoquinone-based algae scavengers are insoluble and therefore are not soluble in water. Therefore, in order to be uniformly applied to the water system, it was necessary to primarily dilute the naphthoquinone-based algae scavengers using organic solvents such as dimethyl sulfoxide (DMSO) solution. Most organic solvents such as DMSO used in conventional naphthoquinone-based compositions for removing harmful algae exhibit ecotoxicity at high concentrations, and therefore, have a problem that there is a possibility of ecosystem disturbance to a certain extent. In addition, the conventional naphthoquinone-based composition requiring an organic solvent entails an increase in additional transportation means and infrastructure for the primary dilution of the solvent, which acts as a factor lowering the economic feasibility of the harmful algae control operation.

### SUMMARY

The present disclosure relates to a naphthoquinone derivative capable of selectively destroying only harmful algae to remove the harmful algae, and a composition and control method for controlling harmful algae using the same. The existing naphthoquinone-based harmful algae removal substance requires an organic solvent to dissolve a powdery substance, but the present disclosure provides a water-soluble naphthoquinone derivative composition that can be used by directly dissolving powder in on-site water by enhancing water solubility without using an organic solvent, a method of preparing the same, a water-soluble composition for controlling harmful algae, a method for controlling large-scale harmful algae, and an automated system for artificial intelligence monitoring, removal, and prevention of large-scale harmful algae.

In an aspect, a method of preparing a water-soluble naphthoquinone derivative composition is provided. The method includes: reacting a 1,4-naphthoquinone compound with N,N-diethylethylenediamine to obtain an intermediate product of [Chemical Formula 2]; and reacting an intermediate product of [Chemical Formula 2] with hydrochloric acid to obtain a compound of [Chemical Formula 1].

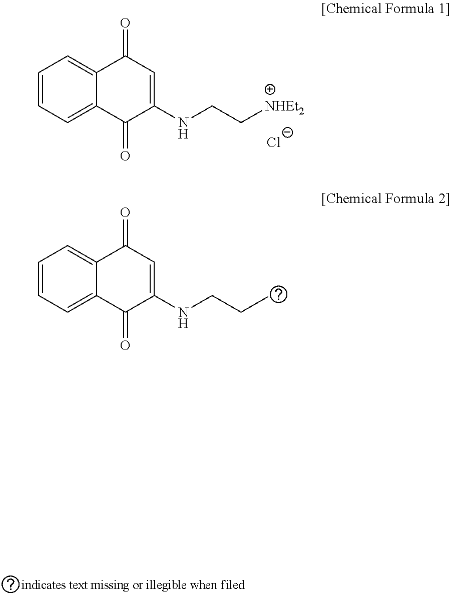

The intermediate product of [Chemical Formula 2] may be produced by adding the N,N-diethylethylenediamine to a mixture in which methanol is mixed with the 1,4-naphthoquinone compound and reacting the mixture, and the compound of [Chemical Formula 1] is produced by mixing and reacting a mixed solution of hydrochloric acid and diethyl ether with a mixed solution of the intermediate product of [Chemical Formula 2] and diethyl ether.

In another aspect, a water-soluble naphthoquinone derivative composition is provided.

The water-soluble naphthoquinone derivative composition represented by the following [Chemical Formula 1].

In another aspect, a water-soluble composition for controlling harmful algae is provided. The water-soluble composition for controlling harmful algae includes: a composition containing the water-soluble naphthoquinone derivative composition according to claim 3 as an active ingredient.

The water-soluble naphthoquinone derivative composition may be prepared by the method of preparing a water-soluble naphthoquinone derivative composition as described above.

The harmful algae controlled by the water-soluble composition for controlling harmful algae may be selected from the group of algae consisting of cyanobacteria, diatomites, dinoflagellates and acicularites.

The cyanobacteria algae may be selected from the group consisting of microcystis, dolichospermum, and aphanizomenon, the dinoflagellate algae may be selected from the group consisting of cochlodinium polykrikoides, alexandrium tamarens, prorocentrum, and akashiwo sanguinea, and the acicular algae may be heterosigma akashiwo.

The water-soluble composition for controlling harmful algae may be prepared in any one formulation of powders, granules, capsules, and liquids.

In another aspect, a method for controlling large-scale harmful algae is provided. The a method includes: spraying the water-soluble composition for controlling harmful algae according to any one of claims 4 to 7 onto a water area in which large-scale harmful algae occurs or a sign of occurrence is observed without an organic solvent.

The water-soluble composition for controlling harmful algae may be sprayed in the form of any one of powder, granular, and capsule or in the form of an aqueous solution.

The water-soluble composition for controlling harmful algae may be sprayed onto a large-scale water area using any one of a remotely controlled unmanned vessel and an unmanned aerial vehicle.

In another aspect, an automated system for artificial intelligence monitoring, removal, and prevention of large-scale harmful algae is provided. The automated system includes: a monitoring device installed in or near a large-scale water area where there is a history of occurrence of harmful algae or a sign of occurrence is expected, and monitoring the occurrence of harmful algae; an unmanned spraying device of any one or more of an unmanned vessel and an unmanned aerial vehicle equipped with a device for spraying and treating the water-soluble composition for controlling harmful algae according to any one of claims 4 to 7 and remotely controlled according to a remote control; and a control device receiving the monitored result from the monitoring device, determining whether the harmful algae occurs, and controlling the unmanned spraying device to perform spraying treatment when the harmful algae occurs according to a set standard.

### BRIEF DESCRIPTION OF THE DRAWINGS

FIG. 1 is a test result of algal effect on various microalgae of a composition according to one embodiment of the present disclosure.

FIG. 2 is a biodegradability test result of a composition according to another embodiment of the present disclosure.

### DESCRIPTION OF EXEMPLARY EMBODIMENTS

Embodiments of the present disclosure for achieving the above object will be described with reference to the accompanying drawings. In the description, the same reference numerals will be used to describe the same components of which a detailed description will be omitted in order to allow those skilled in the art to understand the present disclosure.

Although a singular form is used in the present description, it may include a plural form as long as it is opposite to the concept of the present disclosure and is not contradictory in view of interpretation or is used as a clearly different meaning. It should be understood that “include”, “have”, “comprise”, “be configured to include”, and the like, used in the present description do not exclude presence or addition of one or more other characteristic, component, or a combination thereof.

Hereinafter, the present disclosure will be reviewed in more detail.

There is a problem in that copper sulfate or copper organic compounds, which have been conventionally used as algicide of harmful algae, have excellent control effect on harmful algae, but cause secondary pollution problems due to toxicity and bioconcentration, and also cause ecosystem disturbance since due to the inability to selectively control algae.

On the other hand, the conventional composition for controlling harmful algae including naphthoquinone derivatives presented for selective algae control against harmful algae does not dissolve well in water, and therefore, requires organic solvents such as a dimethyl sulfoxide (DMSO) solution was required. The organic solvents such as DMSO exhibit ecotoxicity at high concentrations, which has the potential to disturb an ecosystem to a certain extent, and also has a problem of cost increase due to an increase in additional facilities for the primary dilution of the solvent.

The present disclosure relates to a naphthoquinone-based composition capable of selectively destroying and removing only harmful algae as a solution to the above-described problem, a derivative thereof, and a method for controlling large-scale harmful algae using a composition. In the present disclosure, since water solubility is enhanced by using a salt, it is possible to directly dissolve and use powder in on-site water without using an organic solvent. According to an embodiment of the present disclosure, it is possible to remove harmful algae only by spraying a composition onto a water system once, and there is no sludge generation and no additional management is required. In addition, due to its high water solubility, it can be manufactured and utilized in various forms such as powder, capsule, granule, and liquid form for convenience of use. Accordingly, it is possible to maximize ecotoxicity stability by the organic solvent, and at the same time increase the economic feasibility by reducing the cost without the use of an organic solvent, and remove harmful algae from a large-scale water area with simple equipment.

In one example of the present disclosure, it is basically applicable to a large-scale harmful algae generating water area (freshwater/seawater), but various uses are possible depending on the purpose. In particular, since it can be used regardless of a water depth, weather, and environmental factors of the applied water system, it can be applied to or used in large-scale lakes, rivers, lakes, ecological ponds, aquaculture farms, water purification plants, and various facilities. In particular, in order to efficiently perform algae removal in the large-scale water area, it can be combined with unmanned ships and unmanned aerial vehicle (drone) technologies. In particular, since a pesticide spraying technology using drones has already reached the commercialization level, the pesticide spraying technology can be combined with these technologies to expect a great synergy effect. In addition, the pesticide spraying technology can be used as a real-time algae monitoring and prevention technology by being combined with the field-installed artificial intelligence automatic microalgae monitoring and spraying device technology.

Meanwhile, in the present disclosure, the ‘harmful algae’ refers to algae that inhabit freshwater or seawater and adversely affect the aquatic environment and economic activities by causing, for example, green algae and/or red algae phenomenon.

[Water-Soluble Naphthoquinone Derivative Composition and Method of Preparing the Same]

A water-soluble naphthoquinone derivative composition and a method of preparing the same according to an example of the present disclosure will be described. In this case, the method of preparing a water-soluble naphthoquinone derivative composition will be first described, and then the water-soluble naphthoquinone derivative composition will be described.

Method of Preparing Water-Soluble Naphthoquinone Derivative Composition

First, a method of preparing a water-soluble naphthoquinone derivative composition according to an example of the present disclosure will be described. The method for preparing a water-soluble naphthoquinone derivative composition described below may be a method for preparing a water-soluble composition for controlling harmful algae described below. In addition, the preparing method described below is an example of a method of preparing a water-soluble naphthoquinone derivative composition according to an example of the present disclosure, and the preparing manufacturing method can be changed through various modifications by those skilled in the art.

In the method of preparing a water-soluble naphthoquinone derivative composition according to an example, the water-soluble naphthoquinone derivative composition may be prepared by reacting a 1,4-naphthoquinone compound with N,N-diethylethylenediamine to obtain an intermediate product of the following [Formula 2] and reacting an intermediate product of the following [Chemical Formula 2] with hydrochloric acid to obtain a compound of the following [Chemical Formula 1]. The intermediate product of the following [Chemical Formula 2] is 2-((2-(diethylamino)ethyl)amino)naphthalene-1,4-dione.

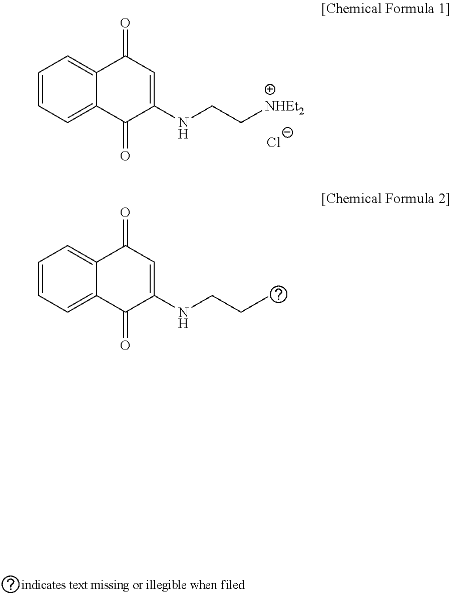

For example, in one example, the intermediate product of [Chemical Formula 2] may be produced by adding the N,N-diethylethylenediamine to a mixture in which methanol is mixed with a 1,4-naphthoquinone compound and reacting the mixture. In addition, the compound of [Chemical Formula 1] may be produced by mixing and reacting a mixed solution of hydrochloric acid and diethyl ether with a mixed solution of the intermediate product of [Chemical Formula 2] and diethyl ether.

### Example

Hereinafter, an example of a process of preparing a water-soluble naphthoquinone derivative composition or a water-soluble composition for controlling harmful algae according to an example of the present disclosure will be described in detail. The specific embodiment below is only one example, and the scope of the present disclosure cannot be naturally limited thereto.

A preparing process according to a specific embodiment is represented by a chemical formula as follows.

First, a process of obtaining the intermediate product of [Chemical Formula 2], 2-((2-(diethylamino)ethyl)amino)naphthalene-1,4-dione from a 1,4-naphthoquinone compound will be described. N,N-diethylethylenediamine (1.00 mL, 6.0 mmol) is added to a well-stirred solution of 1,4-naphthoquinone compound 1 (indicated as ‘1’ in Chemical Formula above, 1.00 g, 6.0 mmol) in methanol (MeOH) (60 mL) at room temperature. When the reaction mixture is stirred at room temperature for 12 hours, a dark brown precipitate is produced. The resulting mixture is concentrated under vacuum and filtered through a plug of silica gel with a ratio of methanol/dichloromethane of 1:15 (methanol/dichloromethane=1:15). The filtrate is concentrated under vacuum, diluted with diethyl ether (Et₂O) and filtered through filter paper. The filtrate is concentrated under vacuum to sufficiently provide pure compound 2-0 (indicated as ‘2-0’ in the Chemical Formula above), i.e., 2-((2-(diethylamino)ethyl)amino)naphthalene-1,4-dione (1.42 g, yield 82%).

Next, a process of obtaining the final product of [Chemical Formula 1], 2-((2-(diethylamino)ethyl)amino)naphthalene-1,4-dione HCl (represented by ‘2-0-HCl’ in the above Chemical Formula) or 2-((1,4-dioxo-1,4-dihydronaphthalen-2-yl)amino)-N,N-diethylethan-1-aminium chloride from the intermediate product of [Chemical Formula 2], 2-((2-(diethylamino)ethyl)amino)naphthalene-1,4-dione (represented by compound 2.0 in the above Chemical Formula) will be described. Diethyl ether ((CF₂H₅)₂O to Et₂O) (for example, 800 mL) is added to a flask filled with 2-((2-(diethylamino)ethyl)amino)naphthalene-1,4-dione (for example, 8 g, 29.3 mmol) represented by [Chemical Formula 2] at room temperature. After 10 minutes, a solution (for example, (2M, 44 mL, 3 equiv)) in which hydrochloric acid (HCl) is dissolved in diethyl ether (Et₂O) at room temperature (rt) is added to the above solution (solution in which diethyl ether (Et₂O) is added to 2-((2-(diethylamino)ethyl)amino)naphthalene-1,4-dione). When the mixture is vigorously stirred for, for example, 1 hour, precipitates are filtered off, washed with diethyl ether (Et₂O), and dried under the reduced pressure, so 2-((2-(diethylamino)ethyl)amino)naphthalene-1,4-dione HCl) is produced.

In this case, 2-((2-(diethylamino)ethyl)amino)naphthalene-1,4-dione HCl salt) is slightly dissolved in ethanol (EtOH), 1,4-dioxane, and well dissolved in dimethyl sulfoxide (DMSO). Therefore, diethyl ether (Et₂O) is recommended for washing.

¹H NMR of 2-0 HCl (400 MHz, D2O): 8.02 (dd, J=7.6, 1.16 Hz, 1H), 7.94 (dd, J=7.8, 1.2 Hz, 1H), 7.83 (td, J=7.5, 1.3 Hz, 1H), 7.74 (td, J=7.6, 1.3 Hz, 1H), 5.79 (s, 1H), 3.73 (q, J=6.3 Hz, 2H), 3.48 (t, J=6.3 Hz, 2H), 3.34-3.32 (m, 4H), 1.32 (t, J=7.3 Hz, 6H).

Water-Soluble Naphthoquinone Derivative Composition

A water-soluble naphthoquinone derivative composition according to an example of the present disclosure is represented by [Chemical Formula 1].

In one example, the water-soluble naphthoquinone derivative composition represented by [Chemical Formula 1] may be prepared according to the example of the above-described preparing method. The above-described preparing method is an example of a method of preparing a water-soluble naphthoquinone derivative composition according to an example of the present disclosure, and should not be construed as limiting the scope of rights of the water-soluble naphthoquinone derivative composition according to one example of the present disclosure.

[Water-Soluble Composition for Controlling Harmful Algae]

Next, the water-soluble composition for controlling harmful algae according to another example of the present disclosure will be described.

The water-soluble composition for controlling harmful algae according to one example includes the above-described water-soluble naphthoquinone derivative composition as an active ingredient. That is, the composition for controlling harmful algae is a substance containing the water-soluble naphthoquinone derivative composition represented by [Chemical Formula 1] as an active ingredient. In this case, the composition for controlling harmful algae may be prepared according to the above-described preparing method or by various modifications.

For example, in one example, the water-soluble naphthoquinone derivative composition, which is an active ingredient of the water-soluble composition for controlling harmful algae, may be prepared according to one of the above-described methods for preparing a water-soluble naphthoquinone derivative composition.

In addition, in one example, the water-soluble composition for controlling harmful algae may be prepared in any one formulation of, for example, powders, granules, capsules, and liquids. The water-soluble composition for controlling harmful algae may also be prepared in other types of formulations not mentioned.

For example, in one example, the harmful algae controlled by the water-soluble composition for controlling harmful algae may be selected from the group of algae consisting of cyanobacteria, diatoms, dinoflagellates, and raphidophyceae.

For example, the cyanobacteria algae may be selected from the group consisting of microcystic, dolichospermum, and aphanizomenon.

In addition, the dinoflagellate algae may be selected from the group consisting of cochlodinium polykrikoides, alexandrium tamarens, prorocentrum, and akashiwo sanguinea.

Furthermore, the raphidophyceae algae may be heterosigma akashiwo.

[Method for Controlling Large-Scale Harmful Algae]

Next, a method for controlling large-scale harmful algae according to one example of the present disclosure is will be described.

The method for controlling large-scale harmful algae according to one example may be performed by spraying any one of the examples of the above-mentioned water-soluble composition for controlling harmful algae onto the water area in which the large-scale harmful algae occurs or signs of occurrence are observed without an organic solvent For example, the composition for controlling harmful algae may be in the formulations of, for example, granules, powders, capsules, etc., but is not limited thereto.

For example, in one example, the water-soluble composition for controlling harmful algae may be sprayed in the form of any one of powder, granular, and capsule or in the form of an aqueous solution.

For example, in another example, it is possible to spray the water-soluble composition for controlling harmful algae onto a large-scale water area using any one of a remotely controlled unmanned aerial vehicle and an unmanned aerial vehicle.

### Implementation Example

For example, when the number of harmful algae cells is generated at a level of 5×10⁵ cells/mL in a 1000-ton reservoir, the water-soluble composition for controlling harmful algae represented by [Chemical Formula 1] prepared according to an embodiment to be described later and about 430 g of powder are mixed with on-site water, and then evenly sprayed onto a water layer in which algae occur. Green algae may be controlled down to the algae advisory level (5,000 cells/mL) or less within a few days or a week.

[Automated System for Artificial Intelligence Monitoring, Removal, and Prevention of Large-Scale Harmful Algae]

Next, an automated system for artificial intelligence monitoring, removal, and prevention of large-scale harmful algae according to another example of the present disclosure will be described. The automated system for artificial intelligence monitoring, removal, and prevention of large-scale harmful algae according to an example of the present disclosure is configured to include a monitoring device, an unmanned spraying device, and a control device.

A monitoring device is installed in or near a large-scale water area where there is a history of occurrence of harmful algae or a sign of occurrence is expected, and monitors the occurrence of harmful algae. For example, the monitoring device may includes an image device to photograph a large-scale water area according to setting and perform artificial intelligence or automated monitoring in a manner of reading the captured image, but is not limited thereto.

Next, the unmanned spraying device may be any one or more of an unmanned aerial vehicle and an unmanned aerial vehicle that are remotely controlled. In this case, each of the unmanned ship and the unmanned aerial vehicle is provided with an automated spraying processor which is a device for spraying the water-soluble composition for controlling harmful algae according to any one of the above-described examples according to the remote control.

In addition, the control device receives the monitored result from the monitoring device, determines whether harmful algae occurs, and controls the unmanned spraying device when harmful algae occur according to a set standard so that the spraying is performed. For example, the control device is configured as a server system to control the monitoring device installed in a plurality of areas, and may perform automation of artificial intelligence monitoring, removal, and prevention of large-scale harmful algae for a plurality of areas.

According to the present disclosure, it is possible to remove the harmful algae in the water system. Since the water solubility of the naphthoquinone-based algae removal substance is greatly improved compared to the existing technology, a separate organic solvent is not required. Not only the ease of use of naphthoquinone substances for removal of harmful algae is increased, but also the cost reduction effect and the risk factors of disturbance of the ecosystem of the solvent are solved. In addition, as shown in the experimental results to be described later, the substance of the present disclosure itself is safe for the ecosystem, and is proved to be a biodegradable substance, and therefore, can be applied in the field in a large-scale water area. In addition, it is possible to selectively control only harmful algae occurring due to abnormal growth of harmful algae (blue-green algae, flagellum, etc.) in ponds, reservoirs, lakes, lakes and marshes, streams, rivers, coasts, and offshores, so it can be very usefully used to prevent the occurrence of harmful algae in freshwater or seawater and to prevent water pollution. In addition, because it can remove harmful algae with only a very small amount, it can be smoothly combined with IT technologies such as artificial intelligence automatic spraying and drone grafting.

[Algal Effect Experiment]

An algal effect experiment on various microalgae including harmful algae was conducted using the composition according to the present disclosure.

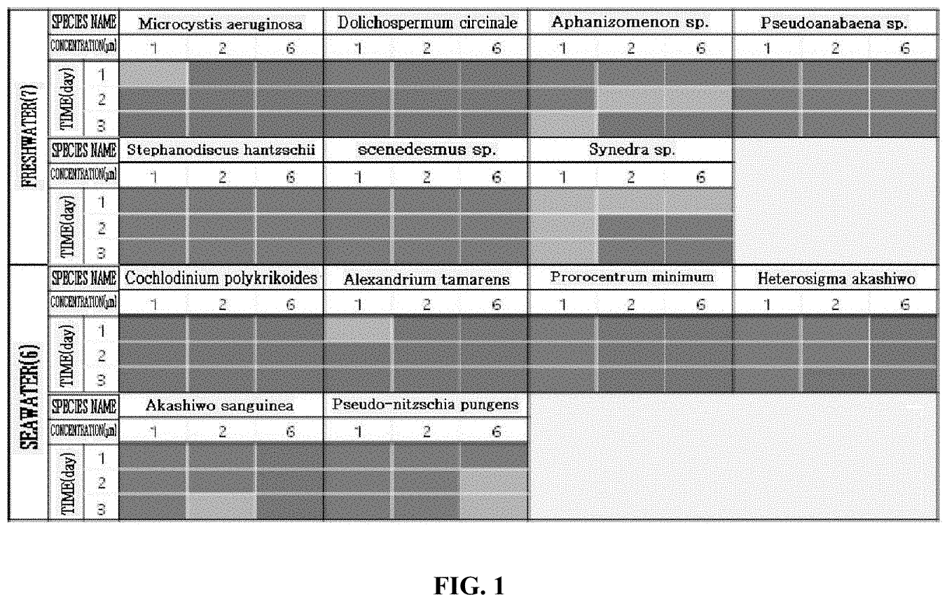

FIG. 1 is a test result of algal effect on various microalgae of a composition according to one embodiment of the present disclosure.

The harmful algae algal effect at various inoculation concentrations of the compounds according to an example of the present disclosure was measured. To this end, freshwater harmful algae as treated group include blue-green algae, cyanobacteria microcystis aeruginosa, dolichospermum circinale, aphanizomenon sp., and pseudoanabaena sp., diatoms, stephanodiscus hantzschii, and synedra sp., and green algae, scenedesmus sp., and seawater harmful algae includes trichodesmium sp., alexandrium tamarens, cochlodinium polykrikoides, prorocentrum minimum, and heterosigma akashiwo which are blue-green algae, and pseudo-nitzschia pungens fungens which is diatoms. The culture conditions are shown in [Table 1] below. [Table 1] shows the culture medium and culture conditions of major algae-inducing algae.

TABLE 1

| Algae | Medium (pH) | Water temperature | Light quantity | Light quantity cycle | Others |
|---|---|---|---|---|---|
| Cyanobacteria | CB (pH 9) | 20~25° C. | 50 μmol/E² · S | 24 hr Light:12 hr Dark | Stationary, shake culture |
| Diatoms | DM (pH 6.9) | 15~20° C. | 50 μmol/E² · S | 12 hr Light:12 hr Dark | Stationary culture |
| Green algae | C (pH 7) | 20~25° C. | 50 μmol/E² · S | 12 hr Light:12 hr Dark | Stationary, shake culture |
| Dinoflagellates | F/2 (pH 8) | 20~25° C. | 50 μmol/E² · S | 12 hr Light:12 hr Dark | Stationary culture |
| Raphidophyceae | F/2 (pH 8) | 20~25° C. | 50 μmol/E² · S | 12 hr Light:12 hr Dark | Stationary culture |

Reviewing the details of the experiment, in the case of freshwater species, 5 mL of each culture line was prepared so that the cell density was 5.0×10³-9.0×10⁴ cells/mL, and in the case of seawater species, was prepared so that the cell density was 1.0×10²-1.0×10⁴ cells/mL. After preparing each culture, the compounds specified as [Chemical Formula 1] prepared according to Examples were inoculated at various concentrations, and are treated so that the final concentrations were 1, 2, and 6 μM, respectively. Then, the compositions were cultured under the culture conditions of [Table 1], and cultured under continuous light conditions and observed for 72 hours. Counting of the number of cells was performed through an SR-chamber or a hemocytometer under an upright or inverted microscope, and by counting the number of cells after 24 hours, 48 hours, and 72 hours, the reduction ratio of cells compared to before treatment, that is, algal activity (%) was calculated using the following [Equation 1].

Algal activity (%)=(1−Tt/Ct)×100 [Equation 1]

In this case, in [Equation 1], T represents the density of cells after treating the compound, C represents the density of cells not treating the compound, and t represents the culture time.

Based on the above [Formula 1], the algal activity of the compound was expressed as 60% or less, 60 to 80%, 80% or more. When the algal activity reached 80% within 48 hours at any compound concentration, it was determined that there was an effect at a concentration higher than that and the high concentration experiment was stopped.

The experimental results will be described with reference to FIG. 1. Referring to FIG. 1, the composition of [Chemical Formula 1] according to the present disclosure shows killed 90% or more of the blue-green algae, microcystis and dolichospermum at a concentration of 1 μM or more, which are major algae causative species in freshwater. In addition, in the case of aphanizomenon belonging to the same blue-green algae, 80% or more of the algae effect was shown at a concentration of 2 μM or more. In addition, the algal effect of cochlodinium polykrikoides which is a major red tide dinoflagellate that causes damage almost every year in the coast of Korea showed an algal effect of more than 90% from a concentration of 1 μM or more, and produced paralytic shellfish, and the algal effect of alexandrium tamarence, which damages fish and shellfish farms, was more than 90% at a concentration of 1 μM or more. In addition, for heterosigma akashiwo, which frequently causes red tides around the world, the algal effect was observed in more than 90% at a concentration of 2 μM or more, and for akashiwo sanguinea, the algal effect was observed in more than 80% at a concentration of 6 μM or more.

On the other hand, the algal effect was found to be insignificant for diatoms and green algae. In the case of Synedra, the algal effect was observed to 70% at 1 μM on the second day, but cell growth was observed again. In particular, in the case of the green algae, Scenedesmus, the algal effect was hardly observed. In general, in the case of the green algae, there are many useful organisms and almost no damage has been reported, so the composition according to the present disclosure has little effect on these species, while it was confirmed that there is an excellent algae effect against harmful algae that cause big problems by inducing green algae and red algae phenomena at home and abroad.

[Ecotoxicity Test]

An experiment was conducted to evaluate the ecotoxicity of the composition according to the example of the present disclosure.

In order to investigate the effect of the compound according to an example of the present disclosure, that is, a composition according to [Chemical Formula 1] on the ecosystem, biotoxicity evaluation indicator organisms such as luminescent bacteria (vibrio fischeri) and daphnia (Daphnia magna) was used to evaluate the ecotoxicity.

First, in the case of the luminescent bacteria, an experiment was conducted according to the water pollution process test method, and as a sample for ecotoxicity evaluation, the compound specified in Example 1 was added to the blue-green algae, microcystis aeruginosa (10,488 cells/mL) at a concentration of 1 μM, and then, the supernatant was taken after 24 hours, 48 hours, and 72 hours and the luminescent bacteria were injected. Thereafter, the relative luminescence of the treated group compared to the control group was measured to confirm the luminescence reduction effect, and thus, the toxicity of each sample was evaluated.

Next, in the case of the Daphnia, an experiment was conducted according to the water pollution process test method, and as the sample for the ecotoxicity evaluation, the compound specified in Example 1 was added to the blue-green algae, microcystis aeruginosa (10,488 cells/mL) at a concentration of 1 μM, and then, the supernatant was taken after 24 hours, 48 hours, and 72 hours and the number of individuals with swimming inhibition was counted after 24 hours.

As a result of the experiment, in the case of the luminescent bacteria in the sample added with the composition represented by [Chemical Formula 1] prepared according to the Example, the relative luminance did not differ from that of the control group (Table 2), and in the case of Daphnia, there were no subjects with swimming inhibition. Therefore, since the ecotoxic effect was not shown at the applied concentration of 1 μM of this compound, this compound is determined to be a suitable substance for application to aquatic ecosystems such as reservoirs, rivers, and lakes. The following [Table 2] showed the relative luminescence of the luminescent bacteria of the sample after a certain time has elapsed after the addition of the algal substance.

TABLE 2

| | After 24 hours | After 48 hours | After 72 hours |
|---|---|---|---|
| Control group | 100% | 100% | 100% |
| Treated group^{a} | 100% | 100% | 100% |

^{a}A sample added at a final concentration of 1 μM was used as a target sample for toxicity evaluation after a certain period of time.

[Experiment on Biodegradability of Algal Substances]

An experiment was conducted to evaluate the ecotoxicity of the composition according to the example of the present disclosure.

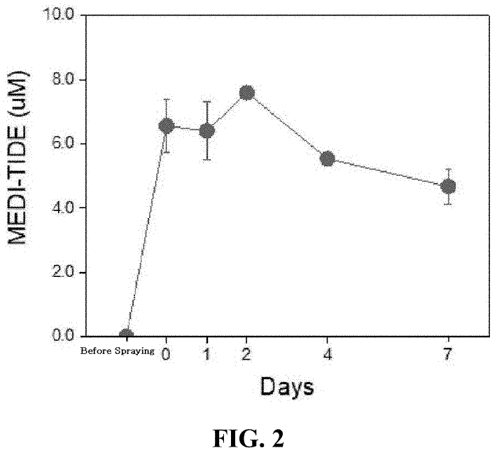

FIG. 2 is a biodegradability test result of a composition according to another embodiment of the present disclosure.

An experiment was performed to evaluate the biodegradability in the water system of the compound of [Chemical Formula 1] according to the example of the present disclosure. First, on-site water from the Jungnangcheon stream in Seoul was collected and sprayed so that the concentration of the substance was 6.5 μM, and then acclimatized to a constant temperature condition of 20° C. The experimental volume was 350 mL, and samples were taken at intervals of 1-3 days, and 5 mL was taken per one time. This sample was filtered with a GF/F filter and stored frozen, and then melted at room temperature for analysis, filtered again with a 0.45 μm nylon filter, and then proceeded under the following analysis conditions.
Equipment: Agilent 1290 Infinity (UHPLC-DAD)
Column: Eclipse Puls-C19 (4.6 mm*100 mm*3.5 μm)
Column temperature: 40° C.
Mobile phase: Isocratic=>A: 0.1% phosphoric acid in D.W, B: ACN=70:30
Analysis time: 7 min
Flow rate: 1.0 mL/min
Detector: DAD Detector (measurement waveform: 260 nm)
Injection volume: 1 μL

FIG. 2 illustrates the biodegradability test results. As a result of the experiment, it was shown that the concentration of the algae substance was maintained at the input concentration level until the first 2 days, but decreased thereafter and decreased to 70% of the initial input concentration on the 7th day. Combining the results of the algal effect experiment of the [Chemical Formula 1] composition prepared according to the example, most of the target algae showing algal activity showed a large effect within 3 days, so it is determined that the biodegradation properties of this substance do not affect the algae removal efficacy. In addition, in general, the physiological activity of bacteria is proportional to the water temperature. In fact, in a high temperature environment in summer where harmful algae thrive, this biodegradation process will be smoother. Therefore, this substance does not permanently remain in the aquatic ecosystem, and it is determined that there is no risk in the aquatic ecosystem due to bioaccumulation or retention.

The accompanying drawings and the above-mentioned exemplary embodiments have been illustratively provided in order to assist in the understanding of those skilled in the art to which the present invention pertains rather than limiting a scope of the present invention. In addition, exemplary embodiments according to a combination of the above-mentioned configurations may be obviously implemented by those skilled in the art. Therefore, various exemplary embodiments of the present invention may be implemented in modified forms without departing from an essential feature of the present invention. In addition, a scope of the present invention should be interpreted according to claims and includes various modifications, alterations, and equivalences made by those skilled in the art.

The present disclosure relates to a water-soluble niphthoquinone derivative composition and a method of preparing the same, and has industrial applicability because it can remove and prevent harmful algae.

According to the present disclosure, unlike the existing naphthoquinone-based harmful algae removal substances that require an organic solvent to dissolve the powdery substance, it is possible to directly dissolve and use powder in on-site water by enhancing water solubility without the use of an organic solvent. Accordingly, according to one example of the present disclosure, it is possible to selectively destroy and remove only harmful algae by directly spraying composition onto on-site water without a solvent or without the use of an organic solvent.

According to one example of the present disclosure, economic feasibility and convenience have been increased because a separate solvent is not required and, for example, an algicide can be directly dissolved in on-site water, and damage such as ecotoxicity that may be caused by a solvent can be reduced

In addition, according to one example of the present disclosure, a harmful algae removal substance, which can be easily dissolved in on-site water without the use of an organic solvent, has superior convenience compared to the existing substances when applied to the field, and thus can have advantages in transportation and movement of the substance. Furthermore, the convenience of using these substances can be used as a very important core technology for removing large-scale algae.

It is obvious that various effects that are not directly stated according to various exemplary embodiments of the present invention may be derived by those skilled in the art from various configurations according to the exemplary embodiments of the present invention.

1. A method of preparing a water-soluble naphthoquinone derivative composition, comprising:
reacting a 1,4-naphthoquinone compound with N,N-diethylethylenediamine to obtain an intermediate product of [Chemical Formula 2]; and
reacting an intermediate product of [Chemical Formula 2] with hydrochloric acid to obtain a compound of [Chemical Formula 1].

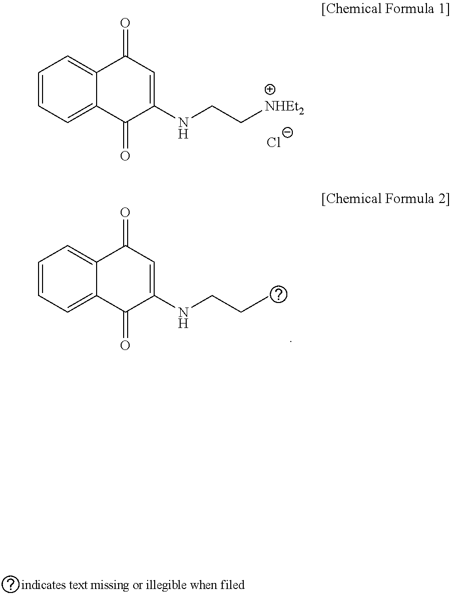

2. The method of claim 1, wherein the intermediate product of [Chemical Formula 2] is produced by adding the N,N-diethylethylenediamine to a mixture in which methanol is mixed with the 1,4-naphthoquinone compound and reacting the mixture, and
the compound of [Chemical Formula 1] is produced by mixing and reacting a mixed solution of hydrochloric acid and diethyl ether with a mixed solution of the intermediate product of [Chemical Formula 2] and diethyl ether.

3. A water-soluble naphthoquinone derivative composition represented by the following [Chemical Formula 1]

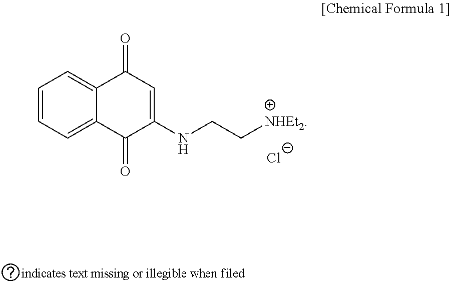

4. A water-soluble composition for controlling harmful algae, comprising:
a composition containing the water-soluble naphthoquinone derivative composition according to claim 3 as an active ingredient.

5. (canceled)

6. The water-soluble composition for controlling harmful algae of claim 4, wherein the harmful algae controlled by the water-soluble composition for controlling harmful algae is selected from the group of algae consisting of cyanobacteria, diatomites, dinoflagellates and acicularites.

7. The water-soluble composition for controlling harmful algae of claim 6, wherein the cyanobacteria algae is selected from the group consisting of microcystis, dolichospermum, and aphanizomenon,
the dinoflagellate algae is selected from the group consisting of cochlodinium polykrikoides, alexandrium tamarens, prorocentrum, and akashiwo sanguinea, and
the acicular algae is heterosigma akashiwo.

8. The water-soluble composition for controlling harmful algae of claim 4, wherein the water-soluble composition for controlling harmful algae is prepared in any one formulation of powders, granules, capsules, and liquids.

9. A method for controlling large-scale harmful algae, comprising:
spraying the water-soluble composition for controlling harmful algae according to claim 4 onto a water area in which large-scale harmful algae occurs or a sign of occurrence is observed without an organic solvent.

10. The method of claim 9, wherein the water-soluble composition for controlling harmful algae is sprayed in the form of any one of powder, granular, and capsule or in the form of an aqueous solution.

11. The method of claim 10, wherein the water-soluble composition for controlling harmful algae is sprayed onto a large-scale water area using any one of a remotely controlled unmanned vessel and an unmanned aerial vehicle.

12. An automated system for artificial intelligence monitoring, removal, and prevention of large-scale harmful algae, comprising:
a monitoring device installed in or near a large-scale water area where there is a history of occurrence of harmful algae or a sign of occurrence is expected, and monitoring the occurrence of harmful algae;
an unmanned spraying device of any one or more of an unmanned vessel and an unmanned aerial vehicle equipped with a device for spraying and treating the water-soluble composition for controlling harmful algae according to claim 4 and remotely controlled according to a remote control; and
a control device receiving the monitored result from the monitoring device, determining whether the harmful algae occurs, and controlling the unmanned spraying device to perform spraying treatment when the harmful algae occurs according to a set standard.
